(19) United States
(12) Patent Application Publication
(10) Pub. No.: US 2026/0306542 A1
(43) Pub. Date: Oct. 8, 2026

(54) Targeted Immune Cell Therapy Utilizing TRAIL and TNF Mediated Apoptosis

(71) Applicant: Northeastern University, Boston, MA (US)

(72) Inventors: Tania KONRY, Boston, MA (US); Matthew SULLIVAN, Medford, MA (US)

(21) Appl. No.: 19/481,721

(22) PCT Filed: Jun. 3, 2024
(86) PCT No.: PCT/US2024/032321

§ 371 Date: Nov. 5, 2025

Related U.S. Application Data
(60) Provisional application No. 63/470,820, filed on Jun. 2, 2023.

(51) Int. Cl. A61K 35/17; A61K 31/7088; A61K 38/00; A61K 40/11; A61K 40/15; A61K 40/31; A61K 40/42; A61K 45/06; C07K 14/525; C07K 14/705; C07K 16/30; C12N 5/0783; C12N 15/86; G01N 33/50

(52) U.S. Cl. CPC A61K 35/17; A61K 31/7088; A61K 38/00; A61K 40/11; A61K 40/15; A61K 40/31; A61K 40/42; A61K 45/06; C07K 14/525; C07K 14/70503; C07K 16/30; C12N 5/0636; C12N 5/0646; C12N 15/86; G01N 33/5011; G01N 33/505; C07K 2317/31; C12N 2510/00; G01N 2500/10

(57) ABSTRACT

The present technology provides a genetically modified immune cell expressing a chimeric antigen receptor (CAR) and also expressing an elevated level of a ligand for a tumor necrosis factor (TNF) receptor. The genetically modified cell takes advantage of dual pathways for killing cancer cells, cellular pathogens, and cells infected with viruses. The CAR-mediated pathway promotes killing action by perforin and granzymes released by cytotoxic T lymphocytes and natural killer cells, as well as the TNF receptor (i.e., death domain) mediated target cell killing by these same cells. Further, the present technology, especially when combined with single cell image analysis, microfluidics based cell sorting, and transcriptomic analysis makes it possible to adapt genetic modifications to cells of a patient, in response to significant patient to patient variation in gene expression of T cells and NK cells.

### CROSS REFERENCE TO RELATED APPLICATIONS

This application claims the priority of U.S. Provisional Application No. 63/470,820 filed 2 Jun. 2023 and entitled “Targeted Immune Cell Therapy Utilizing TRAIL and TNF Mediated Apoptosis”, the whole of which is hereby incorporated by reference.

### STATEMENT REGARDING FEDERALLY SPONSORED RESEARCH OR DEVELOPMENT

This invention was made with government support under Grant No 5R01GM127714-04 awarded by the National Institutes of Health. The Government has certain rights in the invention.

### BACKGROUND

Targeted cellular therapies for cancer treatment, such as CAR-NK and CAR-T cell treatments, show some promise and have been approved for clinical use. However, these treatments are not universally effective for patients, and possess potential side effects that can present their own health risk to patients. There remains a need to develop safer and more effective uses of CAR-NK and CAR-T cells to treat cancer and infectious diseases.

### SUMMARY

The present technology provides genetically modified immune cells, such as CAR-T and CAR-NK cells, with superior efficiency of killing target cells, such as cancer cells. A genetically modified lymphocyte model was developed that activates the tumor necrosis factor (TNF) pathway to kill cancer cells in combination with checkpoint inhibitors. The modified immune cells kill cancer cells more efficiently, and are expected to reduce resource demands, reduce side effects, and improve patient recovery rates.

One aspect of the present technology provides a genetically modified immune cell expressing a chimeric antigen receptor (CAR) and also expressing an elevated level of a ligand for a tumor necrosis factor (TNF) receptor. The genetically modified cell takes advantage of dual pathways for killing cancer cells, cellular pathogens, and cells infected with viruses. The CAR-mediated pathway promotes killing action by perforin and granzymes released by cytotoxic T lymphocytes and natural killer cells, as well as the TNF receptor (i.e., death domain) mediated target cell killing by these same cells. Further, the present technology, especially when combined with single cell image analysis, microfluidics based cell sorting, and transcriptomic analysis makes it possible to adapt genetic modifications to cells of a patient, in response to significant patient to patient variation in gene expression of T cells and NK cells.

Another aspect of the present technology is a kit containing the genetically modified cell described above and one or more bispecific antibodies or aptamers having a first binding specificity for a tumor antigen and a second binding specificity for an immune cell.

Yet another aspect of the present technology is a kit containing one or more viral vectors for genetically modifying an immune cell to express a CAR and to elevate an expression level of a ligand for a TNF receptor, and instructions or one or more reagents for carrying out the genetic modification.

Still another aspect of the present technology is a method of producing a genetically modified cell. The method includes: (a) harvesting immune cells from a subject; (b) optionally screening the harvested immune cells for their ability to kill tumor cells of the subject and selecting harvested immune cells having desired function; and (c) genetically modifying the harvested or screened and selected immune cells for use in anti-tumor therapy for the subject. Even another aspect of the present technology is a method of enhancing the killing of tumor cells in a subject in need thereof. The method includes administering a plurality of the genetically modified cells described above to the subject, whereby killing of tumor cells in the subject is enhanced compared to the absence of the genetically modified cells.

The technology can also be summarized in the following list of features.
1. A genetically modified immune cell expressing a chimeric antigen receptor (CAR) and an elevated level of a ligand for a tumor necrosis factor (TNF) receptor.
2. The genetically modified immune cell of feature 1, wherein the cell is a T-lymphocyte or a natural killer cell.
3. The genetically modified immune cell of feature 1 or 2, wherein the ligand for a TNF receptor is selected from the group consisting of TRAIL, FASL, TNF-α, LT, RANKL, APRIL, BAFF, LIGHT, and VEGI.
4. The genetically modified immune cell of any of the preceding features, wherein the CAR comprises an amino acid sequence of a tumor antigen selected from the group consisting of CD-19, NY-ESO-1, mesothelin, PSA, MART-1, MART-2, Gp100, tyrosinase, p53, ras, MUC1, SAP-1, survivin, CEA, Ep-CAM, Her2, BRCA1/2, and combinations thereof.
5. The genetically modified immune cell of any of the preceding features, wherein the cell further expresses a soluble immune modulator or a soluble immune checkpoint inhibitor.
6. The genetically modified immune cell of feature 5, wherein the soluble immune modulator or soluble immune checkpoint inhibitor is selected from the group consisting of anti-CTLA-4, anti-PD-1, anti-PDL-1, anti-LAG-3, anti-TIM 3, anti-B7-H3, anti-ICOS, IDO inhibitors, 4-1BB, anti-CD47, anti-B7-H4, anti-OX-40, anti-TIGIT, and anti-CD160, and combinations thereof.
7. The genetically modified cell of any of the preceding features, that has been produced by a method comprising:
(a) harvesting immune cells from a subject;
(b) optionally screening the harvested immune cells for their ability to kill tumor cells of the subject and selecting harvested immune cells having desired function; and
(c) genetically modifying the harvested or screened and selected immune cells for use in anti-tumor therapy for the subject.
8. The genetically modified cell of feature 7, wherein step (b) is performed, and wherein the screening comprises analysis of individual harvested immune cells for their ability to kill tumor cells when paired in aqueous microdroplets or cell spheroids in a microfluidic device, and optionally wherein the screening comprises transcriptomic analysis by sequencing of RNA from said harvested immune cells collected from the aqueous microdroplets or cell spheroids.
9 The genetically modified cell of feature 7 or 8, wherein step (b) is performed, and wherein the selecting comprises use of a sorting module in the microfluidic device.
10. The genetically modified cell of any of features 7-9, wherein step (c) comprises transducing the cell with a viral vector comprising a gene for expressing said CAR and a gene for modifying expression of said TNF receptor ligand.
11. A kit comprising the genetically modified cell of any of the preceding features and one or more bispecific antibodies or aptamers having a first binding specificity for a tumor antigen and a second binding specificity for an immune cell.
12. A kit comprising one or more viral vectors for genetically modifying an immune cell to express a CAR and to elevate an expression level of a ligand for a TNF receptor, and instructions or one or more reagents for carrying out the genetic modification.
13. A method of producing a genetically modified cell, the method comprising:
(a) harvesting immune cells from a subject;
(b) optionally screening the harvested immune cells for their ability to kill tumor cells of the subject and selecting harvested immune cells having desired function; and
(c) genetically modifying the harvested or screened and selected immune cells for use in anti-tumor therapy for the subject.
14. The method of feature 13, wherein step (b) is performed, and wherein the screening comprises analysis of individual harvested immune cells for their ability to kill tumor cells when paired in aqueous microdroplets or cell spheroids in a microfluidic device, and optionally wherein the screening comprises transcriptomic analysis by sequencing of RNA from said harvested immune cells collected from the aqueous microdroplets or cell spheroids.
15. The method of feature 13 or 14, wherein step (b) is performed, and wherein the selecting comprises use of a sorting module in the microfluidic device.
16. The method of any of features 13-15, wherein step (c) comprises transducing the cell with a viral vector comprising a gene for expressing said CAR and a gene for modifying expression of said TNF receptor ligand.
17. A method of enhancing killing of tumor cells in a subject in need thereof, the method comprising administering the genetically modified cell of any of features 1-10 to the subject, whereby killing of tumor cells in the subject is enhanced.
18. The method of feature 17, further comprising producing the genetically modified cell by the method of any of features 13-16.
19. The method of feature 17 or 18, further comprising administering a soluble immune checkpoint inhibitor or a soluble immune modulator to the subject.
20. The method of any of features 17-19, further comprising administering to the subject one or more bispecific antibodies or aptamers having a first binding specificity for a tumor antigen and a second binding specificity for an immune cell.

### BRIEF DESCRIPTION OF THE DRAWINGS

FIG. 1A shows the layout of a microfluidic device for forming aqueous microdroplets in an oil stream, co-encapsulating immune cells and target cells in microdroplets, optionally storing and observing the microdroplets in a microwell array, and sorting and harvesting cells based on fluorescence. FIG. 1B shows details of the sorting junction (panel (1)), and droplet docking stations (panels (2) and (3)).

FIG. 2A shows a schematic illustration of a droplet sorting junction based on cell fluorescence. FIG. 2B shows a schematic illustration of a protocol for separating active NK cells from inactive NK cells. FIG. 2C shows a schematic illustration of a protocol for separating serial killer NK cells from less active NK cells.

FIG. 3A shows a schematic illustration of a protocol for separating activated T cells based on an intracellular Ca²⁺ transient. FIG. 3B shows a representation of how an antigen can be presented to a CD8+ T cell to activate it using an MHC tetramer loaded with the antigen. FIG. 3C shows two intracellular calcium signals, each indicating a sorted, activated T cell. FIG. 3D shows the time course of an intracellular calcium signal in a CD8+ T cell (left side of pair) activated by contact with antigen.

FIG. 4, left, shows the layout of a microfluidic device for forming, docking, observing, and collecting cell spheroids containing cancer cells and immune cells. The middle panel shows a higher magnification image of the docking array of the device containing cell spheroids. The right image is a still higher magnification of one spheroid at different times.

FIG. 5 shows the relative expression levels of selected genes under the conditions indicated.

FIG. 6A shows a volcano plot that demonstrates the total quantity of genes upregulated, downregulated, and unchanged between rCHOP and rituximab treated cells. FIG. 6B shows the relative expression levels of selected genes related to lipid metabolism after treatment with rituximab or rCHOP. FIG. 6C shows the relative expression levels of selected gens related to immune activity after treatment with rituximab or rCHOP. FIG. 6D shows the upregulation of the indicated biological processes. FIG. 6E shows the upregulation of the indicated cellular components. FIG. 6F shows the upregulation of the indicated molecular factors.

FIG. 7A shows the relative expression of several genes of different indicated groups of NK cells for each of four donors. FIGS. 7B-1 and 7B-2 shows relative gene expression levels for NK killers vs. non-killers for each of four donors. FIG. 7C shows the log₂ fold change in expression for cytotoxicity-related genes for each of four donors after treatment with a CD3/CD19 bispecific antibody.

FIG. 8A shows the relative expression levels for each of the genes indicated for each of four donors after treatment with a CD3/CD19 bispecific antibody. FIG. 8B shows the log₂ fold change in expression for a series of T cell activation genes for each of four donors after treatment with the bispecific antibody. FIG. 8C shows the log₂ fold change in expression for the indicated genes for each of four donors after treatment with the bispecific antibody.

### DETAILED DESCRIPTION

The present technology enhances the target cell killing ability of immune cells such as NK cells beyond the measures taken in typical chimeric antigen receptor (CAR) therapy. Traditionally, CAR-NK cells are genetically modified to express surface receptors that target specific tumor cell markers, and upon binding release perforin and granzymes to kill the target cell.¹ While highly effective at killing target cells, perforin and granzyme are released into solution and also can kill healthy cells, including immune cells.² Additionally, the granzyme/perforin pathway has been shown to only effectively kill a single target cell, and in subsequent interactions other signaling mechanisms are used.³ This mechanism transition is useful, given the limited quantity of the secreted factors that a cell can carry, and the time required to replenish cellular stores.

The inventors tested CAR-NK and non-transfected, Wt-NK cells prepared through the same procedure, and have shown that CAR-NK cells produce little to no increase in serial killing of target cancer cells. This inability to significantly increase multi-target cell killing can be understood given that CAR cytotoxicity is driven by secretion of granzymes and perforin. The ability of a cytotoxic immune cell to kill multiple target cells is a highly desired feature for a cell-based immune therapy, due to the inability to deliver CAR-NK cells to a tumor at a 1:1 ratio.

The inventors have also studied transcriptomic features of CAR-NK and Wt-NK cells, separated using a microfluidic system into populations that could or could not kill target cancer cells. They found an expected high upregulation of perforin, granzymes, and IFNγ in the CAR-NK killers; however, this was not mirrored in the Wt-NK killers. Instead, minor increases in expression of TNF factors were found. An increase in TNF and TNFSF10 expression by NK cells, and of TNFRSF1A and 21 on target cells in the Wt-killer NK cells compared to non-killers, indicated that cytotoxicity is driven by the TNF pathway.

Additionally, CAR-NK cells showed a much higher expression of certain exhaustion markers, including KLRG1, CD160 and HACVR2 (TIM3). Thus, despite being able to kill their target cell, CAR-NK cells can be expected to lose their activity and have short lifespans in the tumor microenvironment. In contrast, the wildtype NK cells showed relatively low expression of these markers, indicating that these cells are able to kill additional target cells. In both cell types, an upregulation of PDL1 expression was also observed in conditions where target cells were not killed, indicating that this signaling interferes both with CAR-and TNF-mediated apoptosis mechanisms.

In order to supplement the specificity and tumor infiltration of a CAR cell with an enhanced serial killing capability, the present technology provides an immune cell, such as a T lymphocyte or NK cell, that is genetically modified to express a cancer antigen targeting Fc receptor (i.e., a CAR specific for a cancer antigen) and also modified to increase the expression of membrane-bound TNF ligands, such as TNFSF2 or TRAIL. In certain embodiments, these modifications are supplemented with a checkpoint inhibition mechanism, such as a PD1 or PDL1 inhibiting mechanism, either by further genetic modifications of the immune cell or by systematically delivering the checkpoint inhibitor during treatment. TNF signaling provides a slower but highly effective alternative to granzyme and perforin secretion, with reduced collateral damage to nearby healthy tissues.

Further, the scFV portion of a CAR is a powerful tool for bringing immune cells directly to their target cells. For example, the use of anti-CD19 CARs in particular has proven useful for the treatment of lymphoma. Through their CAR binding specificity, CAR-NK cells can effectively find and connect with lymphoma cells. Using TNF-mediated signaling, however, NK cells can produce multiple contact mediated kills without undergoing exhaustion. Combining this with PD1 receptor blocking is expected to greatly enhance the capability of cytotoxic immune cells to kill even resistant cancer cells. This design can be translated to other targets by changing the scFv portion of the CAR to recognize other antigens. Therefore genetically engineered immune cells of the present technology can be a powerful alternative to traditional CAR lymphocytes, particularly for solid tumors.

The present technology provides genetically modified immune cells. The immune cells can be any type of immune cell that is part of the cell-mediated immunity (Th1) response, such as CD8+ T lymphocytes, cytotoxic T lymphocytes, and natural killer (NK) cells, and phagocytes such as macrophages. The immune cells are genetically modified to express an antigen-specific chimeric antigen receptor (CAR) whose antigen-binding specificity is selected to target a desired antigen, such as an antigen specific for cancer cells of a subject who has cancer, or a certain type of cancer, either a solid tumor, or a blood-born cancer, or other cancer. Methods of producing CAR-T cells and CAR-NK cells are well known in the field of immunotherapy. Generally, one or more immune cells, which can be cells of the human or other mammalian subject to be treated, or cells of another individual, is transduced with a viral vector, such as a lentiviral vector or other viral vector. The vector comprises a nucleic acid sequence that encodes the CAR, and also can comprise regulatory sequences such as a promoter, enhancer, and/or sequences that mediate insertion of the vector nucleic acid into the host cell's genome. The structure of CARs is well known. Generally, a CAR has three domains: an extracellular scFv antibody domain that specifically binds the selected antigen, a membrane embedded domain, and a signal transduction domain within the cell, such as CD3zeta from the T cell receptor, which activates the cell upon binding of the scFv domain to its antigen. Any generation of CAR technology, including any of the three generations currently available (Generations 2 and 3 add one or two co-stimulatory domains to the CD3zeta domain of Generation 1) can be used to prepare genetically modified immune cells as described herein. An example of a lentiviral vector for expressing a CAR in T cells can be found at en.vectorbuilder.com/resources/vector-system/pLV_Exp_CAR.html.

The CAR expressed in an immune cell according to the present technology can have binding specificity for any desired antigen. Preferred are cancer cell-specific antigens or antigens specific for cellular pathogens. Examples of suitable cancer cell antigens include CD-19, NY-ESO-1, mesothelin, PSA, MART-1, MART-2, Gp100, tyrosinase, p53, ras, MUC1, SAP-1, survivin, CEA, Ep-CAM, Her2, and BRCA1/2. Examples of antigens specific for cellular pathogens include cell surface proteins, glycoproteins, or oligosaccharides of protozoans, parasites, and bacteria. For these antigens, epitopes to be targeted for binding by the CAR are well known and can be selected from the literature or by other means known to the person of ordinary skill.

A viral vector used to transduce a cytotoxic immune cell according to the present technology contains one or more nucleic acid sequences that encode a membrane-bound TNF receptor ligand, in addition to sequences required for transduction and expressing the CAR. The viral vector can be a lentiviral vector or a gamma retroviral vector, for example. Nucleic acid sequences encoding TNF ligands such as TRAIL, FASL, TNF-α, LT, RANKL, APRIL, BAFF, LIGHT, and VEGI (see Dostert, C., et al., The TNF Family of Ligands and Receptors: Communication Modules in the Immune System and Beyond, Physiol. Rev. 2018, 99, 115-160) can be used, for example. Preferred are TNF ligands such as TRAIL, FASL, TNF-a that bind to a TNF receptor that contains a cytosolic death domain, such as DR4, DR5, Fas, or TNFR1. Some TNF receptor ligands exhibit cross-reactivity that allows them to bind to more than one TNF receptor. The corresponding TNF receptor will be found on the target cell, such as a cancer cell, and preferably is not significantly expressed on normal cells. Nucleic acid sequences encoding TNF ligands, including those described above, and suitable for expression from a viral vector in a cytotoxic immune cell, are well known, and any such sequences can be used. The transgene for expressing the TNF receptor ligand can be positioned in the vector such that it is under control of the same promoter as that which controls CAR expression, or it can follow a different promoter. A variety of promoters are known which can be selected based on their ability to regulate expression in the type of host cell and/or their ability to cause expression of the TNF receptor ligand at a desired level in the host cell. It is preferred to express the TNF receptor ligand at a higher level in the genetically modified immune cell than it may be expressed normally in the absence of transduction by the viral vector. For example, expression of the TNF ligand can be increased by 20%, 50% 100%, 1.5-fold, 2-fold, 3-fold, 4-fold, 5-fold, 10-fold, or more compared to the non-transduced host cell, so that optimal effectiveness in killing of targeted cells is achieved, The viral vector used to transduce a cytotoxic immune cell and express a CAR and a TNF receptor ligand can optionally further include one or more nucleic acid sequences encoding a soluble immune modulator or checkpoint inhibitor. Examples of suitable soluble immune modulators or checkpoint inhibitors include anti-CTLA-4 (e.g., ipilimumab), anti-PD-1 (e.g., nivolumab, pembrolizumab), anti-PDL-1 (e.g., atezolizumab, avelumab, durvalumab), anti-LAG-3, anti-TIM 3, anti-B7-H3, anti-ICOS, IDO inhibitors, 4-1BB, anti-CD47, anti-B7-H4, anti-OX-40, anti-TIGIT, and anti-CD160. Nucleic acid sequences suitable for expressing these checkpoint inhibitors are well known.

The present technology includes the following novel features. (1) Utilization of the TNF pathway for mediating cytotoxicity in lymphocyte-induced killing of cancer cells. (2) Active targeting of tumor antigens by transducing immune cells with a CAR combined with a membrane-bound TNF ligand. (3) Combination with checkpoint inhibitor therapy to enhance killing efficacy. (4) A versatile therapy design that can be easily repurposed and produced for a range of different target cancer cells.

The present technology includes the following unique advantages and improvements over currently available approaches. (1) Targeted immune cells for TNF-mediated tumor cell killing increases the efficiency of immune cells compared to previous designs. This means cancers can be treated in some cases with fewer regimens of therapy. (2) Using TNF mediated apoptosis will help treat cancers that are resistant to the immune pathways used by other therapies. (3) Membrane-bound TNF will not interact with cells that the lymphocyte is not directly in contact with. The treatment is expected to cause reduced damage to healthy tissues, and thus reduced side effects.

Uses of the present technology include as a treatment for cancers with targetable antigens, such as CD19+ lymphomas. This technology also has the capability to target other cells of interest, such as autoreactive immune cells and cells with viral or bacterial infections. Therefore, in addition to treatment of cancer, the technology can be used for treatment of autoimmune and infectious diseases.

### EXAMPLES

### Example 1. Methods for Imaging Cell Interactions in Droplets, Cell Sorting, and Collection

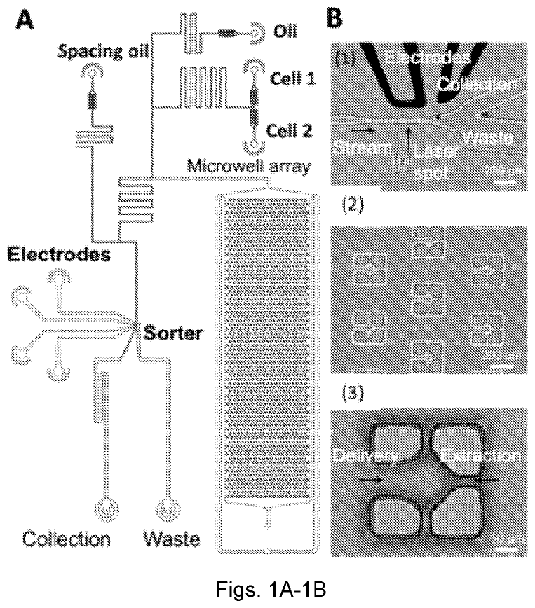

In order to evaluate the phenotypic diversity of cancer cell-immune cell interactions at the single-cell level, a Fluorescence Detection and Droplet Sorting (FADS) was used. The system used a microfluidic device (FIG. 1A) with a high-throughput on-chip droplet docking array capable of analyzing 10,000 events. The docking array contained U-shaped microwells for on-demand trapping, extraction, and incubation of droplets on a single microfluidic chip (FIG. 1B). The integrated FADS system allowed the following. (1) Rapid enrichment of droplets containing user-defined ratios of T-tumor cell pairs, increasing the efficiency of heterotypic co-encapsulation events. (2) Selective sorting of antitumor T cell phenotypes based on classification of activity (i.e., inactive, active (killing single target cell), serial killers (killing two or more target cells in succession), to perform downstream analysis. The recovery of each of the sorted cellular subsets, after functional characterization, for subsequent ‘omics’ level analysis allowed correlation of cell function with the underlying molecular profile (functionomics or transcriptomics) and to distinguish rare cell sub-papulations. (3) In addition, the sorted cellular subsets with anti-tumor activity, after recovery from droplets, can be expanded to produce a cell population with optimal anti-tumor potential.

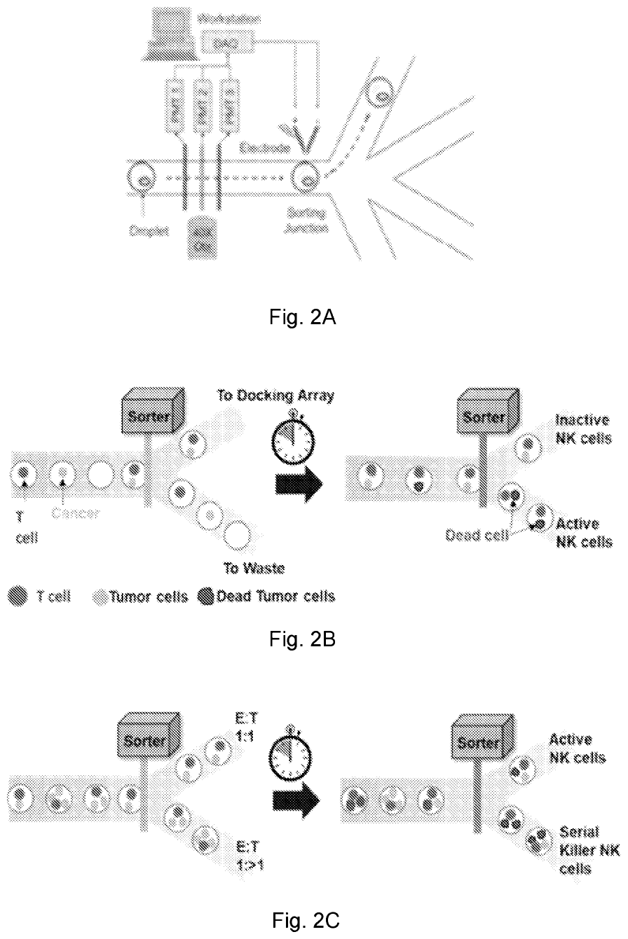

The FADS control system consisted of three main elements: excitation, detection, and sorting (FIG. 2A). The excitation was provided by three different laser lines (Opto Engine, 405 nm, 488 nm, and 561 nm 100 mW laser lines). These allowed for blue (DAPI), green (FITC) and red (dsRed) fluorescence detection) combined in a laser combiner (Doric Lenses), collimated, and connected to the epi-fluorescence port of a standard fluorescence microscope. This system illuminated the sample (a microfluidic droplet in the sorter device) through the microscope objectives. The detection systems included a series of beam-splitters, dichroic mirrors (Semrock), and band-pass emission filters that selectively directed the emission wavelength to three dedicated photomultiplier detectors (PMTs, Hamamatsu). Simultaneously, trans-illumination was available from the trans-illumination port of the microscope, with the addition of an appropriate filter to filter out emission wavelengths from the illumination. The PMTs were wired to an I/O board (National Instruments) with a LabView-based program that monitored the PMT outputs and sent sort pulses when the detected signals were above a user-defined threshold. The pulse originated by the I/O board was amplified by a high-voltage amplifier (Trek Labs.) and connected to the microfluidic device electrodes. The droplet sorting segment of the microfluidic device included a straight channel where spaced droplets traveled single-file at 430 ul/hr, a detection region where the illumination-detection system is focused, and a sorting fork flanked by electrode channels filled with metal electrodes. Droplets were automatically sorted at the outlet based on detection of up to 3 distinct fluorescent markers at 170 droplets/min. As droplets passed single-file through the field-of-view, fluorescent signals were detected with a bank of PMTs, and droplets were guided into 1 of 4 outlet channels using dielectrophoresis (i.e., corresponding to the three fluorophores plus an unlabeled droplet channel). The sorter could run for several hours until the desired number of droplets were collected for analysis. Major sub-system components are described below.

Excitation Lasers. Up to 3 solid-state diode lasers (Opto Engine) were focused in slits across the microfluidic channel corresponding to the excitation wavelengths of the three fluorophores (405 nm, 488 nm, 561 nm). In combination with the fluorescence detection filters, these were selected to be well spectrally separated to allow multiplexed detection with minimal cross-talk between channels. Beams were expanded with cylindrical lenses and then focused to a slit though a 40X× microscope objective at different positions along the channel.

Photomultiplier Tubes (PMTs). A set of 3 PMTs was used to detect the fluorescence signal from droplets. Emitted fluorescence was collected with a second microscope objective, split into 3 detection channels with 50 -50 beam-splitters, and then filtered with appropriate band-pass filters corresponding to each fluorophore (centered at: 405 nm, 488 nm, 561 nm). The output of each PMT was pre-amplified and then acquired with a multi-function data acquisition board (National Instruments) connected to a personal computer. Software running on the PC allowed real-time detection of fluorescent spikes on each of the 3 detector channels. Sorting Junction. After passing through the laser slits, droplets were diverted into collection outlets using dielectrophoresis. A microelectrode was deposited next to the channel at the sorting junction during fabrication and connected to a high voltage, programmable power supply (Stanford Research Systems). Software written in LabVIEW controlled the magnitude and frequency of the AC voltage applied across the electrodes based on droplet fluorescent signature, thereby controlling the magnitude of deflection and outlet channel. The amplitude of the AC potential was selected to be as small as possible to minimize the possibility of shearing the droplets.

Alignment Optics. A white-light source and monochrome CCD camera allowed alignment of the microfluidic chip on the optical system, and alignment of orthogonal illumination of the flow channel with the 3 laser slits and detection optics.

Integration of array with sorter. To sort droplets based on the cell-cell (or cell-drug) interactions, a device was developed that integrates the device with an array of U-shaped microwells. The U-shaped microwells enable on-demand trapping, extraction, incubation, and merging of droplets. This equips the device with the capability to sort droplets depending on the real-time monitored phenotype changes of the cells upon interactions. The cell-encapsulated droplets were first sorted and collected, and then the collected droplets were reinjected into the microwells. This results in the enrichment of cell-encapsulated droplets trapped and incubated in the microwells. A fluorescence detection technique was employed for monitoring the real-time interactions of the cells inside the droplets during an incubation period. The controlled and targeted delivery of cells or drugs was achieved through droplet merging in the microwells, and the extraction of droplets from the microwells was achieved by simply reversing the flow direction. The extracted droplets were then sorted by flowing the droplets through the sorting junction.

Image Acquisition in the array. The phase or fluorescence images of cells in droplets was captured using a Zeiss Axio Observer.Z1 Microscope (Zeiss, Germany) equipped with a Hamamatsu digital camera C10600 Orca-R2, 10-40× objectives, and standard FITC/DAPI/TRITC/FR filters. The microfluidic device containing cells co-encapsulated in droplets was maintained in a humidified microscopic stage-top incubator at 37 °C and 5% C ₂ for the duration of the experiment. All time-lapse images were obtained by automated software control, and the Zen imaging program (Zeiss) was set to travel to specific x-, y-, and z-positions at every 5 minutes for a total period of 6-8 hours for video microscopy. For single cell lysis assays, images were collected at 0 h to determine the initial occupancy of each droplet and every 5 min for 4-12 h. Image processing and analysis was done with ImageJ.

Co-encapsulation of effector T cells (E) and target cells (T). Effector and target cells were co-encapsulated in droplets in various ratios, such as 1:5 to 5:1. “Basal/inactive” T cells were defined as those that did not kill target cells or only killed after prolonged co-encapsulation. “Active” T cells killed one target rapidly, while “hyperactive” T cells (also referred to herein as “serial killers”) killed multiple targets sequentially. See FIGS. 2B, 2C). Since cell loading in droplets is characterized by a Poisson distribution, the large-scale droplet array typically includes multiple E:T ratios in the same experimental run. Typically, a fraction of CD8+ T cells cause the majority of target cell deaths, killing target cells serially, while the rest of immune cells were less cytotoxic. As control, cells were co-incubated at E:T ratios of 1:2, 1:5, and 1:10 in 96-well plates to permit one effector cell to interact with multiple targets sequentially.

Droplet enrichment. Optionally, the T cells were labeled with blue CellTracker CMAC Dye (ThermoFisher), while the target tumor cells (e.g., K562, HL-60, or Raji cells) were labeled with green Calcein AM dye. Then the cells were injected into the chip to generate droplets at a throughput of 200 droplets/sec. The two cell types were introduced through separate inlets to prevent cell interaction prior to co-encapsulation in droplets. The resulting droplets contained various numbers and types of cells. The mixed droplet population was flowed through the sorting system to enrich in droplets that contained one T cell labeled blue and one tumor cell labeled green for loading into the droplet array. Other droplets were considered waste material and removed.

Sorting after imaging in the array. When the docking array was fully occupied by blue +green droplets, droplet generation was stopped and acquisition of dual-color fluorescence images of the array was begun. After incubating cell pairs in the array for 4-16 hrs, the droplets were released, and the sorting gate was set on green+blue channel positive, which selected the droplets containing both blue (T cells) and green (tumor cells). The time-lapse microscopy-based approach allowed profiling of dynamic interaction histories at the single-cell level by quantifying the following parameters: (i) viability of each cell type; (ii) immune-complex formation; (iii) duration and frequency of contact; (iv) rate of association/dissociation; (v) change in motility; and (vi) timing of immune-mediated target cell lysis. In case the T cell did not kill the target cell, which retained its green fluorescence, the T cell was considered inactive. The droplets that did not undergo positive selection, i.e., the droplets separated by negative selection at the second sorting gate, consisted of two populations: (a) only blue cells, when the T (blue) cell has killed the tumor cell (i.e., loss of green), suggesting that there are hyperactive T cells in that population; and (b) only green cells, where the T (blue) cell has died. A minimal death rate of <2% was observed during the duration of the experiments.

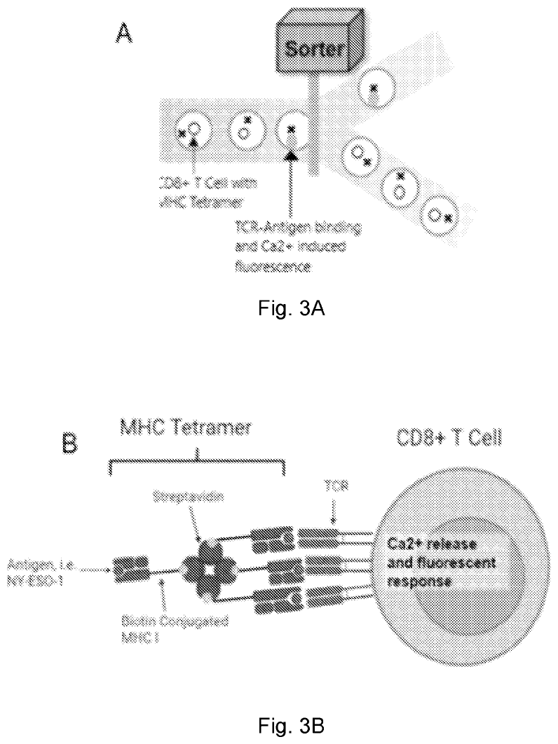

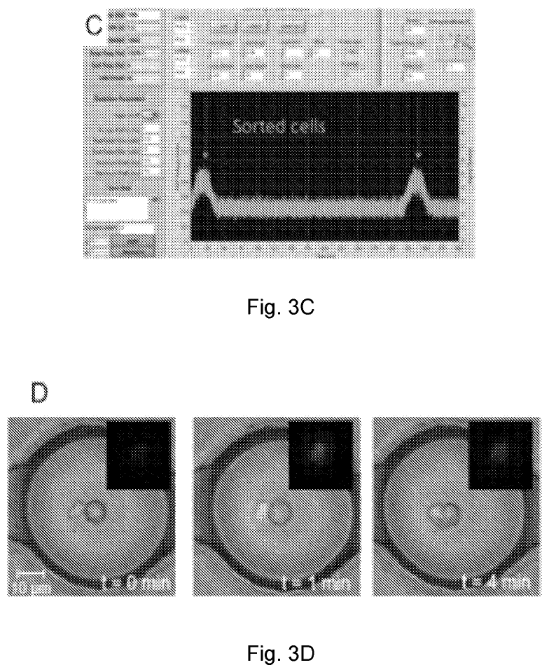

Fluorescence-based droplet sorting of CD8+ T Cells to screen TCR binding affinity. Since the amount of calcium released in a T cell is proportional to the number of T cell receptors (TCRs) bound, a fluorescence threshold was established to indicate T cells with highest antigen affinity. Upon TCR binding to antigen, calcium is released inside the T cell as part of the secondary messenger cascade. This produces a strong fluorescence signal, allowing sorting of antigen-bound T cells in droplets (FIG. 3A). A higher measured calcium response is expected to be associated with a stronger cytotoxic response when T cells are introduced to their corresponding antigen, such as an antigen present on cancer cells.

Droplet Sorting and Collection based on Calcium Signal in CD8+ T Cells. A cancer cell antigen, such as NY-ESO-1 peptides, can be conjugated with MHC tetramers, following established methods. Antigen-induced CD8+ T cells also can be prepared as described in the literature, and mixed with antigen-conjugated MHC tetramers on-chip, and given 10 seconds to mix prior to running through the sorting array (FIG. 3B). An intracellular calcium stain such as Fura-10 AM (AAT Bioquest) can be used to identify the calcium spike following TCR binding to the antigen conjugate. Using the droplet sorting system, the resulting fluorescent signal produced by the calcium release (FIGS. 3C, 3D) activates the sorter and leads to isolation of the activated CD8+ T cell. To further refine the selection of CD8+ T cells based on sensitivity to antigen, the concentration of MHC tetramer can be adjusted, and the threshold of fluorescence signal for sorting also can be adjusted. This can ensure exclusive isolation of T cells with a strong response to the antigen.

### Example 2. Methods for Analyzing Cell Interactions in Cell Spheroids

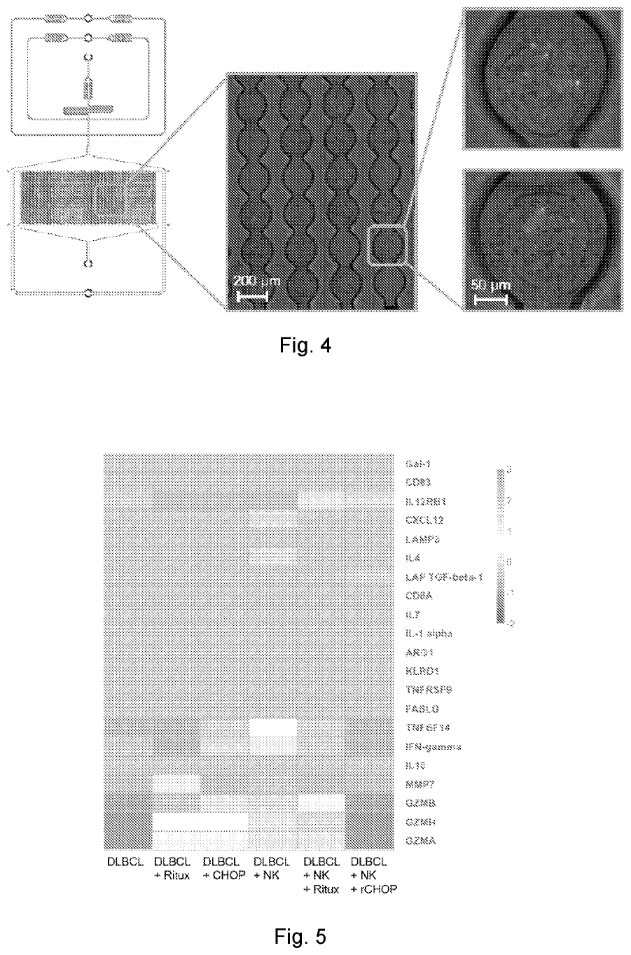

Microfluidic Device Fabrication. Devices were fabricated using standard soft lithography techniques with PDMS (FIG. 4). Silicon wafers were spin coated with SU-8 2075, and UV crosslinked in two stages, one to make the perfusion channels and one to make the device design. Channel heights were made to approximately 120 μm, with perfusion channels an additional 40 μm. Devices were made with 10:1 PDMS and curing agent and bonded to glass slides with a Harrick Plasma Cleaner (Harrick Plasma Inc, Ithaca, NY). Devices were made hydrophobic by perfusing them with AQUAPEL (PPG Industries, Inc, Pittsburgh, PE) through the channels and allowing them to dry.

Cell Culture. Diffuse large B-cells (DLBCL) were provided by Dr. Andrew Evan at Rutgers Cancer Institute, derived from metastatic lymph nodes isolated from patients. DLBCL were cultured in RPMI-1640 (ATCC, Manassas, VA) with 10% FBS and 1% antibotic/antimycotic mixture (Gibco, Waltham, MA). Peripheral blood NK cells were purchased from Stemcell Technologies (Vancouver, BC, Canada). NK cells were thawed the day prior to experiments and rested overnight in RPMI 1640.

Device Filling and Hydrogel Preparation. Alginate stocks were prepared at 4% in distilled water and filtered with a 0.22 μm filter. Vitrogel-RGD High Concentration was purchased from TheWell Biosciences (North Brunswick, NJ). Alginate solution was either mixed with RGD Vitrogel or added alone to glass vials with a stir bar. Cells were pelleted and resuspended in HBSS, then added to alginate while continuously stirring. Alginate was made to 1% w/v final concentration, either without or supplemented with 7.5% v/v RGD+ Vitrogel. After approximately 30 seconds of stirring, 1 mL of the cell suspension was perfused into the microfluidic devices with Tygon microbore tubing (Saint-Gobain Company, Courbevoie, France) using Harvard Apparatus syringe pumps (Holland, MA). Mineral oil with 2% v/v Span-80 surfactant (MilliporeSigma, Munich, Germany) was used to create the aqueous-in-oil droplet emulsions. Once the docking sites of the droplet array were satisfactorily filled, the perfusion of cells and oil was stopped. To crosslink the droplets, 125 mM calcium chloride solution in RPMI 1640 was perfused into the device at a low flow rate, approximately 15 to 50 μL/h. Once the calcium solution displaced all oil from the device, the syringes were replaced with complete media, including the relevant treatment conditions, and perfusion was maintained at 50 μL/h for the remainder of the experiment.

Viability imaging and Cell Preparation. For viability imaging, an Axio Observer microscope equipped with an automated stage and incubation chamber was used while maintaining cells at 37 °C and 5% CO₂. The microscope was also equipped with a light source and filter set for fluorescence of DAPI, EGFP, dsRed and far-red channels. DLBCL were incubated with 10 μM CMAC CellTracker (ThermoFisher) in serum-free media for 45 minutes and washed twice with media prior to pelleting and resuspending in hydrogel. NK cells were incubated with 2 μM CFSE CellTracker (Thermofisher) and resuspended with DLBCL for co-culture conditions. NK cells were loaded with DLBCL at a 1:2 ratio (NK:DLBCL). NK cells were loaded at 5 to 7.5 million cells/mL, while DLBCL were loaded at 10 to 15 million cells/mL. Prior to viability imaging, devices were perfused with 8 μM ethidium homodimer (Biotium, Freemont, CA) for approximately 3 hours. Spheroids were differentiated between low density and high density, with high density having >5 cells/mm².

Immunofluorescence Imaging. For immunofluorescence imaging, a Zeiss LSM 880 confocal microscope was used (Zeiss). The microscope was equipped with 405, 458, 488, 514, 561, 594 and 640 nm excitation lasers, Airyscan superresolution, and spectral unmixing capabilities. For imaging, cells were not labeled with dyes prior to loading spheroids. For DLBCL, the antibodies used were anti-CD47 conjugated to PerCP-Cy 5.5, anti-PDL1 conjugated to Alexa Fluor 594, and anti CD20 conjugated to Brilliant Violet 421 (Biolegend). For NK cells, anti-SIRPα conjugated to FITC, anti-PD1 conjugated to Alexa Fluor 647, and anti-CD16 conjugated to PE were used for labeling (Biolegend). Prior to imaging, devices were infused with antibodies in serum-free RPMI-1640 at 50 μL/h for 2 hours. After antibody labeling, cells were fixed by perfusion with BD fixation buffer for 1 hour, then washed with HBSS via perfusion for 1 hour. Images were taken at 20× magnification. Unlabeled spheroids loaded with an identical cell concentrations were used as controls for autofluorescence.

### Example 3. Secretion and Transcriptomic Analysis of Cell Spheroids

Secretion Analysis. While devices underwent overnight perfusion, the flow-through was collected from individual treatment conditions to analyze secretions released from the DLBCL and NK. These secretions were sent to Olink Proteomics (Uppsala, Sweden) for multiplexed analysis with their Target 96 Immuno-Oncology panel. Of the 96 markers analyzed, 21 displayed notable levels in the collected samples of interest (FIG. 5). One of the markers found secreted by DLBCL alone was lysosome-associated membrane protein 3 (LAMP3), a marker associated with metastasis from lymph nodes. Other factors expressed in this condition were CXCL12 and IL12 receptor (IL12RB1), both factors known to help activate cytotoxic immune cells. The lack of expression of the markers in treated conditions suggests DLBCL take a more resistant phenotype when under stress. The condition with DLBCL and NK displayed the highest levels of granzyme secretions, which were reduced with the addition of treatments. DLBCL+NK+rituximab showed high expression of numerous alternative factors associated with immune cell activation and inflammation. This result is reasonable, as rituximab binding activates the ADCC pathway in NK cells, which should produce a highly active and cytotoxic phenotype. Several of these secretions, however, also have been shown to promote immune cell evasion in late stage cancers, and can lead to immune cell desensitization or exhaustion. These markers were all reduced with the addition of CHOP in the DLBCL+NK+rCHOP condition. This may be related to the anti-inflammatory activity of prednisone in the CHOP cocktail. Instead, Gal-1 and CD83 are found at increased levels. Gal-1 has been correlated to increased metastasis and immune evasion, while CD83 is known to decrease NK cell activation.

Transcriptomic Sequencing from Cell Spheroids. For transcriptomic sequencing, cells were first re-collected from hydrogels. To re-collect cells, a 10 mM sodium citrate solution supplemented with 50 mM EDTA was perfused through the microfluidic device containing cell spheroids at 200 μL/h. The device was kept on the microscope at 37 °C and monitored constantly. Perfusion was performed for approximately 30-45 minutes, until alginate was completely un-crosslinked. Total RNA was extracted from collected cells using the RNeasy Mini Kit (QIAGEN, Venlo, Netherlands), then frozen at −80 °C. Frozen samples were sent to Novogene (Beijing, China) for transcriptomic analysis using a Takara SMART-seq V4 kit for amplification and the Illumina NovaSeq platform for sequencing. Data processing and bioinformatics was provided by Novogene.

Transcriptomic Sequencing of Cells Re-collected from Spheroids. To understand what mechanisms were increasing cytotoxicity in disagreement with the secretomic profile, a transcriptomic analysis was performed on cells in the DLBCL+NK+rituximab and the DLBCL +NK+rCHOP conditions for comparison. Alginate was dissolved with a chelating solution, cells were collected, and RNA extracted via the RNeasy kit. Extracted total RNA was subjected to transcriptomic sequencing.

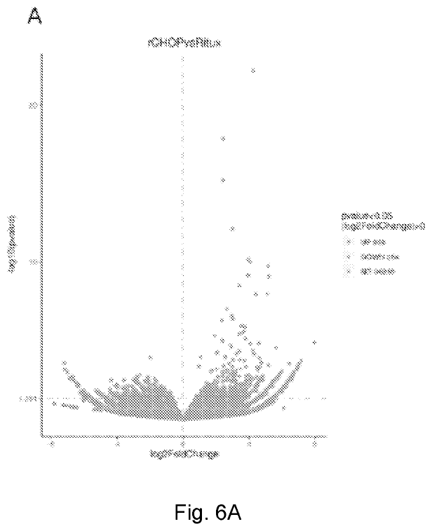

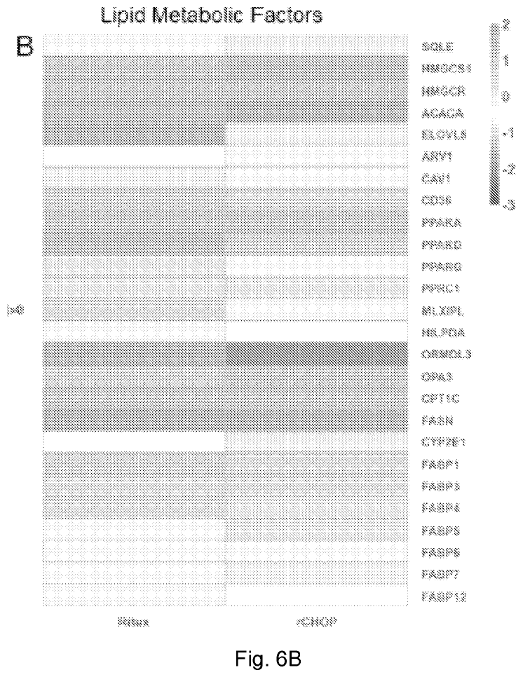

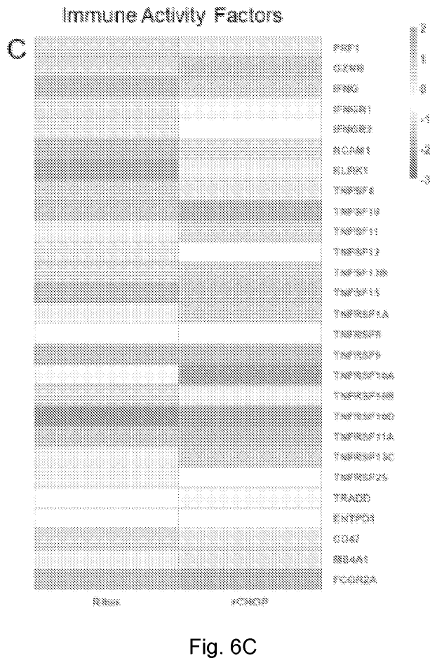

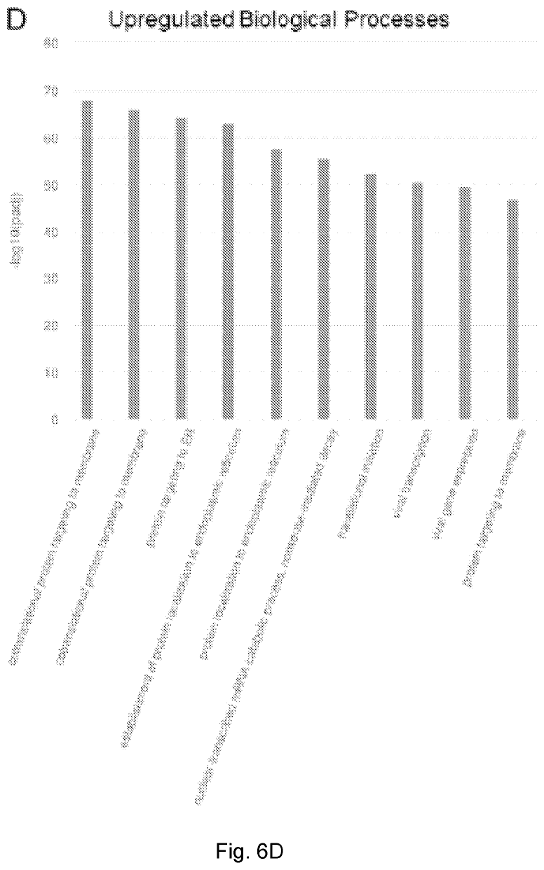

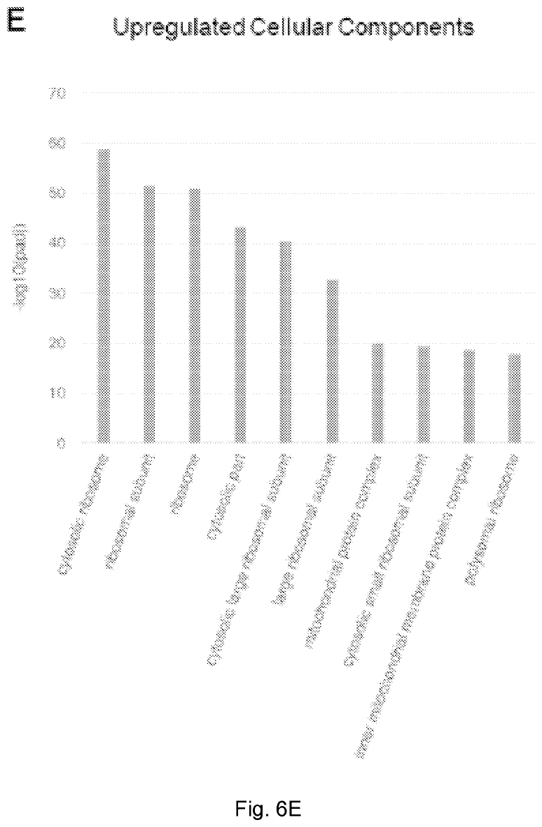

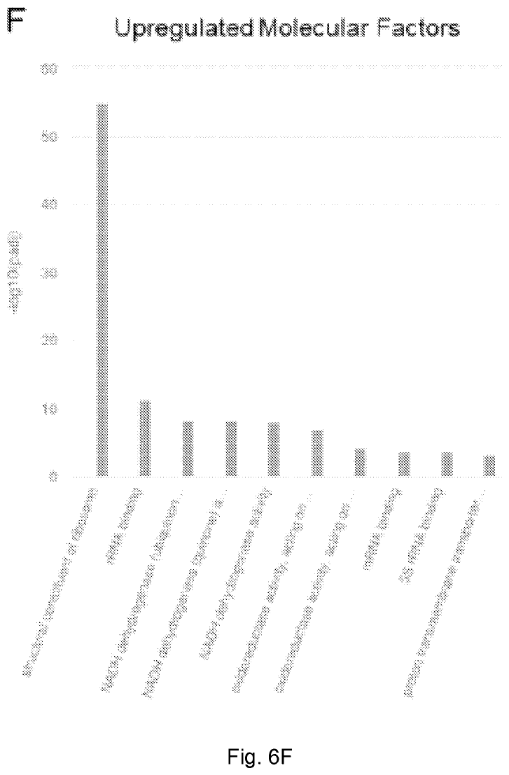

From the overall gene expression comparison of these samples, 608 genes were upregulated and 354 were downregulated in the rCHOP treatment compared to rituximab alone (FIG. 6A). Reviewing the Gene Ontology (GO) analysis of these samples, several genes related to ribosomes were upregulated in rCHOP, both in Cellular Components and in Molecular Processes (FIGS. 6E, 6F). This upregulation insinuates more bioactivity in the rCHOP sample, suggesting potential metabolic and activity shifts in response to CHOP. Looking at expression of the top-expressed ribosomal proteins, and ribosome biogenesis genes, a mixed response was seen for each treatment. Across ribosomal proteins, an average increase in expression was found in rCHOP, except mitochondrial ribosomal proteins, which were found to be slightly higher in rituximab alone treatment. While many biological factors can contribute to ribosomal changes, mitochondrial ribosome gene expression has been linked to resistance in cancer cells. Ribosomal RNA expression has been shown to have a range of consequences in cancer and other diseases, correlating both to cancer resistance and NK cells activity. In addition to ribosome upregulation, protein trafficking and enzymatic activity, such as NADPH and oxidoreductase expression, were found upregulated in rCHOP treated cells. Overall, this insinuates that metabolic activity in the rCHOP treated cells may be elevated, potentially through DLBCL resistance mechanisms to CHOP treatment.

To get more information of the metabolic activity differences between these conditions, expression of genes related to lipid and fatty acid synthesis and metabolism was examined (FIG. 6B). CAV1, CD36, MLXIPL, CPT1C and CYP2E1 were found to be more highly expressed in the cells treated with rituximab alone. Increased lipid metabolic activity has been correlated with cancer resistance, and these markers have specifically been shown to be upregulated in cancers with poor prognosis. Several lipid factors were also upregulated in the rCHOP conditions, including ACACA, HMGCs, FASN and SQLE. These have been associated with lymphocyte activation, and they may be signs of enhanced NK metabolic activity; however enhanced lipid metabolism has also been linked to impaired NK cell function. Several fatty acid binding proteins (FABPs) were also considered, as well as Peroxisome Activator Receptors (PPARs). FABP 5, 7 and 12, and PPARA and D were found at higher levels in rCHOP treated cells. FABP and PPAR expression has been found to correlate to NK cells maintaining critical cytotoxic capabilities in the lymphoma microenvironment. FABPs and PPARs also can contribute to DLBCL resistance, however, and their upregulation may be another sign of treatment resistance.

The expression of several major factors in immune activity and cytotoxicity were also examined (FIG. 6C). Looking at perforin, granzyme and IFNγ expression, little difference was seen between the two populations. However, several differences were noted in the TNF family of receptors and ligands. Of particular interest, TNFSF10 (TRAIL) was upregulated, while TNFSF11 (RANKL) and TNFSF12 (TWEAK) were downregulated. TRAIL is a well known ligand expressed on NK cells and other immune cells that can directly induce apoptosis in target cells. High expression of TNFRSF 10B, a TRAIL receptor that induces apoptosis, in the rCHOP conditions supports TRAIL-mediated apoptosis as a factor in the increase observed in target cell killing.

When comparing the secretomic data (FIG. 5) to the transcriptomic data (FIGS. 6A-6F), there appears to be an increase in cellular activity. The transcriptomic data reveals a more active NK phenotype when treated with rituximab alone, while the transcriptomic data shows increased metabolic activity with rCHOP. Given these results, it seems that the increased metabolic activity can be primarily attributed to the DLBCL response to CHOP. These metabolic changes may sensitize the DLBCL to NK cell ADCC mediated cytotoxicity, without the need of a high inflammatory response. In clinical studies, rCHOP has been shown to have much higher rates of patients with complete responses than patients with rituximab alone.

### Example 4. Transcriptional Profile of Bite Activated CD8+ T-Cell Killers

Analysis of CD8+ T-cell Killers via Granule Exocytosis and Cell Death Pathways.

Formation of an immunological synapse between a bispecific antibody (bsAb) activated CD8+ T cell and a tumor cell is a critical step in target mediated clearance. However, the dynamic interaction induced by the bsAb at the single-cell level is largely unknown. Here, the synaptic conjugation between untreated and bsAb-treated CD8+ T cell and tumor cell co-encapsulations were characterized. Raji cells, a well-studied CD19-positive lymphoma cell line, was used as the target. The selected bsAb (blinatumomab) has an scFv domain specific for CD3 on T cells joined by a glycine-serine linker to another scFv domain specific for CD-19. Single cell analysis revealed significant heterogeneity in the cell contact profiles and killing kinetics within the bsAb treated T cell/Raji cell co-encapsulations.

RNA-seq of F-FADS sorted BITE activated CD 8+ T-cell killers and non-killers from 4 donor samples was performed. A total of 15389±1492 genes for CD 8+ killers and 13209±2632 genes for CD8+ non-killers were identified in these samples, with a threshold of fragments per kilobase of exon model per million reads mapped (FPKM) >0.5. Principle component analysis (PCA) was used to emphasize variation as well as similarity in the data-set.

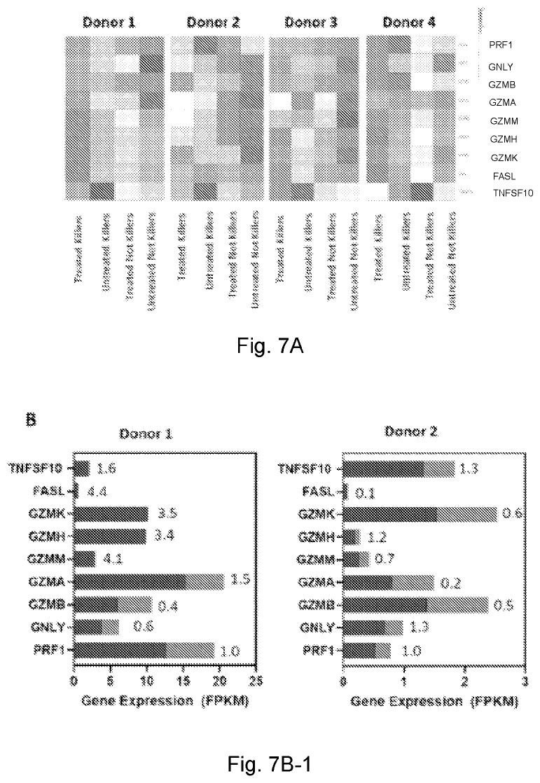

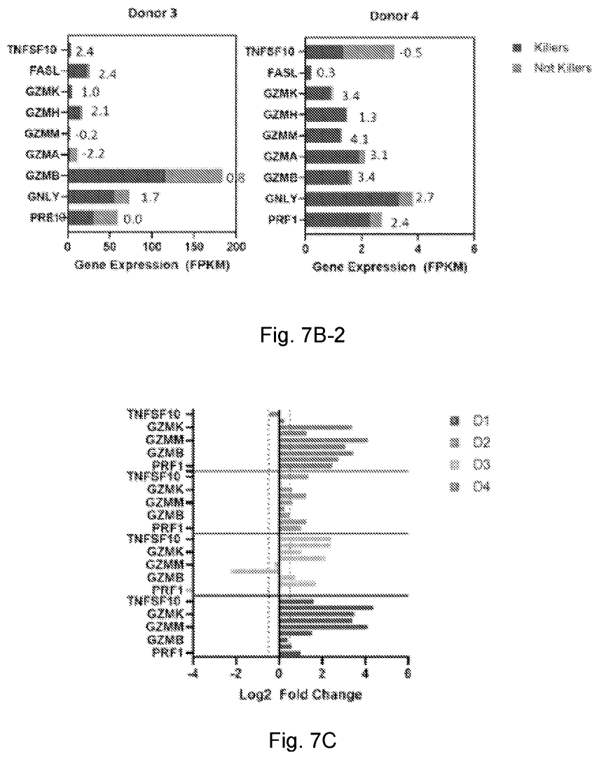

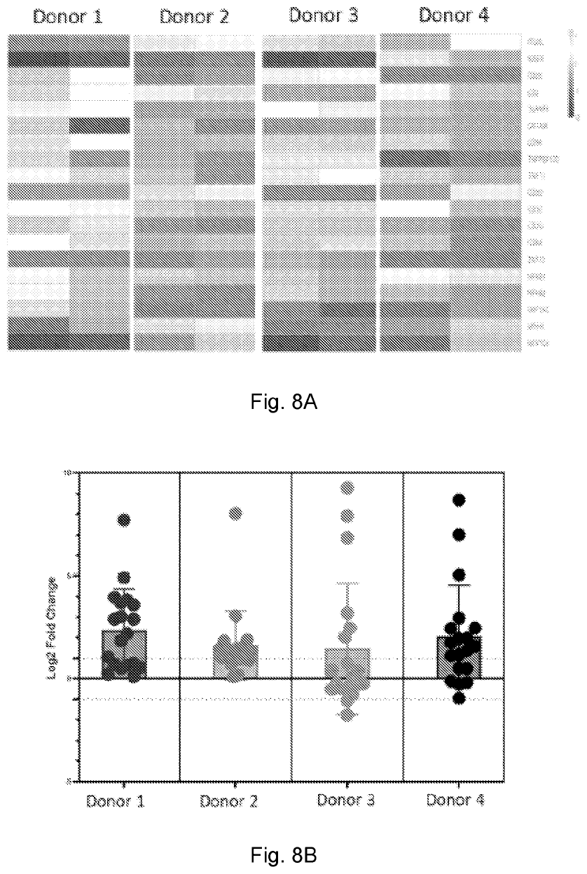

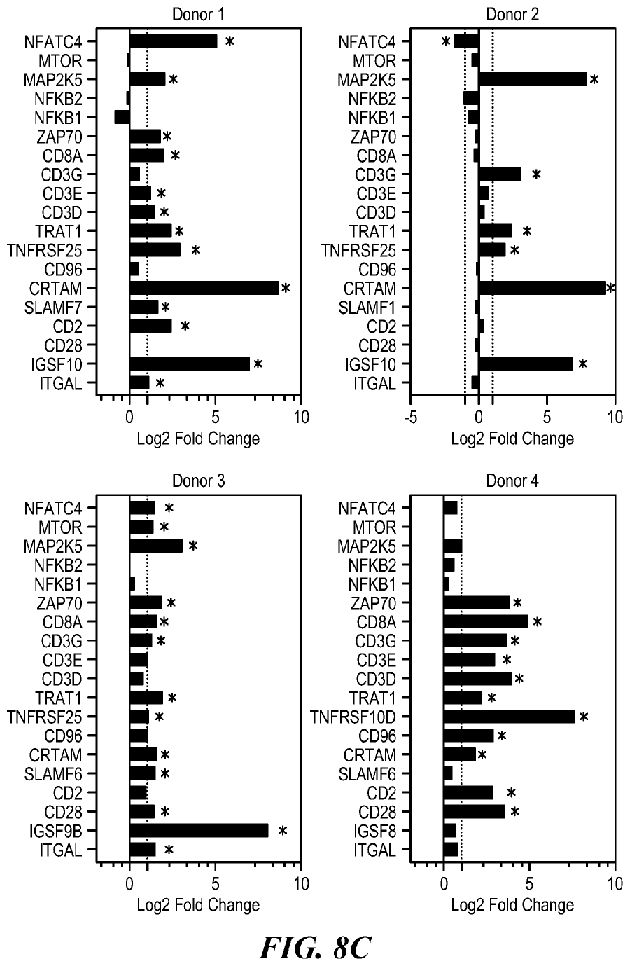

A global representation of BITE activated CD8+ killers and non-killers is shown in the PCA plots (not shown). In this PCA analysis, BITE activated CD8+ T-cell killers and non-killers from all the donors did not form a distinct cluster, suggestive of unique molecular heterogeneity within each donor sample. To explore this observation further, the number of genes uniquely expressed by killers vs. non-killers was evaluated, in addition to the number of genes that were co-expressed (FIGS. 7A, 7B-1, 7B-2, 7C). BITE activated CD8+ T-cell killers displayed more unique genes (mean=2409±257) compared with non-killers (mean=935±231). Overall, there was a statistically significant greater number of unique genes associated with killers vs non-killers. Overall, there were fewer unique genes to each sorted subset compared to the number of co-expressed genes (mean=9791±599). The number of genes common to all killer samples and non-killer samples was similar (9239 and 10,996 respectively).

Next, the differentially expressed gene (DEG) counts for upregulated and downregulated genes were determined for each comparison. Genes were downregulated and upregulated based on log₂ fold-change >1 and log₂ fold-change <−1, respectively, with an adjusted p-value <0.05. The number of upregulated genes (5123±3351) was found to be statistically higher than the number of downregulated genes (1104±928) across all the samples. In total, 6256±4263 genes were found to be dysregulated across all the donors. To determine if the CD8+ T-cell killers sorted using F-FADS possess unique cytolytic capabilities, the gene expression of well-known cytolytic markers was assessed. Cytotoxic T-cells mediate lysis of target cells by exocytosis of specialized cytoplasmic granules containing cytotoxins perforin (PRF1), granzyme B (GZMB) and granulysin (GNLY), and receptor-ligand binding of Fas ligand (FASL) and TNF-related apoptosis-inducing ligand (TRAIL) molecules (ref: www.ncbi.nlm.nih.gov/books/NBK27101/). Cluster analysis of DEGs suggested that the number of cytolytic genes upregulated by BITE activated CD8+ T-cell killers was generally higher compared to non-killers. A statistically significant upregulation (log₂ fold change >1 between CD8+ killers vs. non-killers) was observed in cytolytic genes involved in both exocytosis of lytic proteins and receptor-ligand binding to mediate lysis of target cells. TNFSF10 (TRAIL) and FASL were upregulated to a greater extent compared to cytolytic genes GZMB, GNLY and PRF1. This observation likely reflects the timepoint we selected to F-FADS sort the samples since after 16 h of co-encapsulation of target and effector cells most of the cytoplasmic granule stores are depleted, while the death receptor ligands are likely to be more active.

Also observed was upregulation of a number of other granzymes (GZMK, GZMH, GZMM, and GZMA). Although the main function of these granzymes are presumed to be direct elimination of target cells, the specific mechanism and type of cell death induced varies and is still an active area of investigation. GZMK and GZMA have been shown to induce activation of inflammatory cytokines such as IL-1β, while GZMM has been demonstrated to induce morphological changes associated with microtubule disruption. GZMH is still poorly understand given that some studies have shown that GZMH acts independently of caspases while other studies found GZMH induces caspase-dependent form of cell death.

Interestingly, donor to donor differences were observed in the expression of cytolytic genes. BiTE activated CD8+ T-cell killers from some donors (specifically donors 1 and 3) had low GZMB expression levels and no difference was observed between killers and non-killers. These same donors, however, had statistically significant upregulation in other granzymes (GZMH, GZMA), FASL and TNFSF10. Other donors (specifically donors 2 and 4) had statistically significant upregulation of GZMB in BITE activated CD8+ T-cell killers compared to non-killers, while there was no upregulation in FASL and TNSF10. Furthermore, the change in GZMK, GZMH, GZMM and GZMA between killers and non-killers in these two donors had opposite effects, that is, when one gene was low the other was high. These results suggest donor to donor differences in the mechanism of tumor cell kill, whereby some donors may rely primarily on the release of cytotoxic granules while others more on death receptor-mediated cytotoxicity to kill tumor cells.

### Example 5. Immune Checkpoint Changes in Bite Activated CD8+ T-Cell Killers

Immune checkpoint molecules are inhibitory receptors on the surface of immune cells that ensure appropriate regulation of the immune response. Prominent checkpoint molecules include programmed cell death receptor 1 (PD-1) and CTLA-4 expressed by T cells and other immune cells. Other checkpoint molecules include lymphocyte-activation gene 3 (LAG-3), T-cell immunoglobulin and mucin domain-3 (TIM-3), T-cell immunoreceptor with Ig and ITIM domains (TIGIT) and inducible T-cell co-stimulatory receptor (ICOS). The expression of immune checkpoint molecules can be used to monitor CD8+ T-cell exhaustion.

Changes in expression of immune checkpoint genes were investigated in the 4 CD8+ T cell donors tested in Example 4. Interestingly, CD244 and TIGIT were upregulated in 3 of the 4 donors. CD244 expression has been shown to be a mediator of CD8+ T-cell exhaustion in the setting of persistent antigen exposure, but it has also been shown to be co-expressed on a subset of antigen-experienced effector and effector memory CD8+ T-cells along with other immunoregulatory receptors such as PD-1 but not TIGIT. CD244 has also been shown to be a prominent regulator involved in T cell aging.

In order to better understand if the immune checkpoint changes were potential markers of T-cell exhaustion, a panel of cytokine and chemokine genes was examined. Exhaustion of T-cells is typically accompanied by loss of effector cytokines such as interferon-γ (IFNG), tumor necrosis (TNF) and interleukin-2 (IL2) as well as chemokine receptors CCL2 and CCL5. Increases were observed after CD3/CD19 bispecific antibody treatment of CD8+ T cell killers in IL2, IL7R, IL12RB, IL15, IL18R1, IL18BP, IFNG, and TNF, but not in all donors. Donors 2 and 4 appear to have an overall dampened cytokine response compared to donors 1 and 3. BiTE activated CD8+ T-cell killers from donor 2 and 4 also had increased GZMB, EOMES, CTLA4 and PDCD1 compared to the other donors. Since GZMB is typically lost in advanced stages of exhaustion, these results may reflect an early exhaustion phenotype or a highly activated state.

As used herein, “consisting essentially of” allows the inclusion of materials or steps that do not materially affect the basic and novel characteristics of the claim. Any recitation herein of the term “comprising” can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### References

1. Wang X, Yang X, Yuan X, Wang W, Wang Y. Chimeric antigen receptor-engineered NK cells: new weapons of cancer immunotherapy with great potential. Exp Hematol Oncol. 2022; 11(1): 85. Epub 20221102.doi: 10.1186/s40164-022-00341-7. PubMed PMID: 36324149; PMCID: PMC 9628181.
2. Voskoboinik I, Whisstock J C, Trapani J A. Perforin and granzymes: function, dysfunction and human pathology. Nat Rev Immunol. 2015; 15(6): 388-400. doi: 10.1038/nri3839. PubMed PMID: 25998963.
3. Prager I, Liesche C, van Ooijen H, Urlaub D, Verron Q, Sandstrom N, Fasbender F, Claus M, Eils R, Beaudouin J, Onfelt B, Watzl C. NK cells switch from granzyme B to death receptor-mediated cytotoxicity during serial killing. J Exp Med. 2019; 216(9): 2113-27. Epub 20190703. doi: 10.1084/jem.20181454. PubMed PMID: 31270246; PMCID: PMC 6719417.
4. Dostert C, Grusdat M, Letellier E, Brenner D. The TNF Family of Ligands and Receptors: Communication Modules in the Immune System and Beyond. Physiol Rev. 2019; 99(1): 115-60. doi: 10.1152/physrev.00045.2017. PubMed PMID: 30354964.
5. Sellmer L, Kovacs J, Walter J, Kumbrink J, Neumann J, Kauffmann-Guerrero D, Kiefl R, Schneider C, Jung AD, Behr J, Tufman A. Markers of Immune Cell Exhaustion as Predictor of Survival in Surgically-Treated Early-Stage NSCLC. Frontiers in Immunology. 2022; 13. doi: ARTN 858212 10.3389/fimmu.2022.858212. PubMed PMID: WOS: 000822933000001.
6. Corselli M, Saksena S, Nakamoto M, Lomas W E, 3rd, Taylor I, Chattopadhyay P K. Single cell multiomic analysis of T cell exhaustion in vitro. Cytometry A. 2022; 101(1): 27-44. Epub 20210921. doi: 10.1002/cyto.a.24496. PubMed PMID: 34390166; PMCID: PMC9293072.
7. Montfort A, Dufau C, Colacios C, Andrieu-Abadie N, Levade T, Filleron T, Delord J P, Ayyoub M, Meyer N, Segui B. Anti-TNF, a magic bullet in cancer immunotherapy? J Immunother Cancer. 2019; 7(1): 303. Epub 20191114. doi: 10.1186/s 40425-019-0802-y. PubMed PMID: 31727152; PMCID: PMC 6857159.
8. Freeman A J, Kearney C J, Silke J, Oliaro J. Unleashing TNF cytotoxicity to enhance cancer immunotherapy. Trends Immunol. 2021; 42(12): 1128-42. Epub 20211106. doi: 10.1016/j.it.2021.10.003. PubMed PMID: 34750058.
9. Bremer E. Targeting of the tumor necrosis factor receptor superfamily for cancer immunotherapy. ISRN Oncol. 2013; 2013: 371854. Epub 20130611. doi: 10.1155/2013/371854. PubMed PMID: 23840967; PMCID: PMC 3693168.
10. Albinger N, Hartmann J, Ullrich E. Current status and perspective of CAR-T and CAR-NK cell therapy trials in Germany. Gene Ther. 2021; 28(9): 513-27. Epub 20210322. doi: 10.1038/s41434-021-00246-w. PubMed PMID: 33753909; PMCID: PMC 8455322.
11. Feins S, Kong W, Williams E F, Milone M C, Fraietta J A. An introduction to chimeric antigen receptor (CAR) T-cell immunotherapy for human cancer. Am J Hematol. 2019; 94(S1): S3-S9. Epub 20190218. doi: 10.1002/ajh.25418. PubMed PMID: 30680780.
12. Xie G, Dong H, Liang Y, Ham J D, Rizwan R, Chen J. CAR-NK cells: A promising cellular immunotherapy for cancer. EBioMedicine. 2020; 59: 102975. Epub 20200824. doi: 10.1016/j.ebiom.2020.102975. PubMed PMID: 32853984; PMCID: PMC 7452675.
13. Ebrahimiyan H, Tamimi A, Shokoohian B, Minaei N, Memarnejadian A, Hossein-Khannazer N, Hassan M, Vosough M. Novel insights in CAR-NK cells beyond CAR-T cell technology; promising advantages. Int Immunopharmacol. 2022; 106: 108587. Epub 20220209. doi: 10.1016/j.intimp.2022.108587. PubMed PMID: 35149294.
14. Spiegel J Y, Patel S, Muffly L, Hossain N M, Oak J, Baird J H, Frank M J, Shiraz P, Sahaf B, Craig J, Iglesias M, Younes S, Natkunam Y, Ozawa M G, Yang E, Tamaresis J, Chinnasamy H, Ehlinger Z, Reynolds W, Lynn R, Rotiroti M C, Gkitsas N, Arai S, Johnston L, Lowsky R, Majzner R G, Meyer E, Negrin R S, Rezvani A R, Sidana S, Shizuru J, Weng W K, Mullins C, Jacob A, Kirsch I, Bazzano M, Zhou J, Mackay S, Bornheimer S J, Schultz L, Ramakrishna S, Davis K L, Kong K A, Shah N N, Qin H, Fry T, Feldman S, Mackall C L, Miklos D B. CAR T cells with dual targeting of CD19 and CD22 in adult patients with recurrent or refractory B cell malignancies: a phase 1 trial. Nat Med. 2021; 27(8): 1419-31. Epub 20210726. doi: 10.1038/s 41591-021-01436-0. PubMed PMID: 34312556; PMCID: PMC8363505.
15. Wang X, Diamond D J, Forman S J, Nakamura R. Development of CMV-CD19 bispecific CAR T cells with post-infusion in vivo boost using an anti-CMV vaccine. Int J Hematol. 2021; 114(5): 544-53. Epub 20210924. doi: 10.1007/s12185-021-03215-6. PubMed PMID: 34561840; PMCID: PMC 8475363.
16. Summers C, Wu Q V, Annesley C, Bleakley M, Dahlberg A, Narayanaswamy P, Huang W, Voutsinas J, Brand A, Leisenring W, Jensen M C, Park J R, Gardner R A. Hematopoietic Cell Transplantation after CD19 Chimeric Antigen Receptor T Cell-Induced Acute Lymphoblastic Lymphoma Remission Confers a Leukemia-Free Survival Advantage. Transplant Cell Ther. 2022; 28(1): 21-9. Epub 20211010. doi: 10.1016/j.jtct.2021.10.003. PubMed PMID: 34644605.

What is claimed is:

1. A genetically modified immune cell expressing a chimeric antigen receptor (CAR) and an elevated level of a ligand for a tumor necrosis factor (TNF) receptor.

2. The genetically modified immune cell of claim 1, wherein the cell is a T-lymphocyte or a natural killer cell.

3. The genetically modified immune cell of claim 1, wherein the ligand for a TNF receptor is selected from the group consisting of TRAIL, FASL, TNF-α, LT, RANKL, APRIL, BAFF, LIGHT, and VEGI.

4. The genetically modified immune cell of claim 1, wherein the CAR comprises an amino acid sequence of a tumor antigen selected from the group consisting of CD-19, NY-ESO-1, mesothelin, PSA, MART-1, MART-2, Gp100, tyrosinase, p53, ras, MUC1, SAP-1, survivin, CEA, Ep-CAM, Her2, BRCA1/2, and combinations thereof.

5. The genetically modified immune cell of claim 1, wherein the cell further expresses a soluble immune modulator or a soluble immune checkpoint inhibitor.

6. The genetically modified immune cell of claim 5, wherein the soluble immune modulator or soluble immune checkpoint inhibitor is selected from the group consisting of anti-CTLA-4, anti-PD-1, anti-PDL-1, anti-LAG-3, anti-TIM 3, anti-B7-H3, anti-ICOS, IDO inhibitors, 4-1BB, anti-CD47, anti-B7-H4, anti-OX-40, anti-TIGIT, and anti-CD160, and combinations thereof.

7. The genetically modified cell of any of claim 1, that has been produced by a method comprising:
(a) harvesting immune cells from a subject;
(b) optionally screening the harvested immune cells for their ability to kill tumor cells of the subject and selecting harvested immune cells having desired function; and
(c) genetically modifying the harvested or screened and selected immune cells for use in anti-tumor therapy for the subject.

8. The genetically modified cell of claim 7, wherein step (b) is performed, and wherein the screening comprises analysis of individual harvested immune cells for their ability to kill tumor cells when paired in aqueous microdroplets or cell spheroids in a microfluidic device, and optionally wherein the screening comprises transcriptomic analysis by sequencing of RNA from said harvested immune cells collected from the aqueous microdroplets or cell spheroids.

9. The genetically modified cell of claim 7, wherein step (b) is performed, and wherein the selecting comprises use of a sorting module in the microfluidic device.

10. The genetically modified cell of claim 7, wherein step (c) comprises transducing the cell with a viral vector comprising a gene for expressing said CAR and a gene for modifying expression of said TNF receptor ligand.

11. A kit comprising the genetically modified cell of claim 1 and one or more bispecific antibodies or aptamers having a first binding specificity for a tumor antigen and a second binding specificity for an immune cell.

12. A kit comprising one or more viral vectors for genetically modifying an immune cell to express a CAR and to elevate an expression level of a ligand for a TNF receptor, and instructions or one or more reagents for carrying out the genetic modification.

13. A method of producing a genetically modified cell, the method comprising:
(a) harvesting immune cells from a subject;
(b) optionally screening the harvested immune cells for their ability to kill tumor cells of the subject and selecting harvested immune cells having desired function; and
(c) genetically modifying the harvested or screened and selected immune cells for use in anti-tumor therapy for the subject.

14. The method of claim 13, wherein step (b) is performed, and wherein the screening comprises analysis of individual harvested immune cells for their ability to kill tumor cells when paired in aqueous microdroplets or cell spheroids in a microfluidic device, and optionally wherein the screening comprises transcriptomic analysis by sequencing of RNA from said harvested immune cells collected from the aqueous microdroplets or cell spheroids.

15. The method of claim 13, wherein step (b) is performed, and wherein the selecting comprises use of a sorting module in the microfluidic device.

16. The method of claim 13, wherein step (c) comprises transducing the cell with a viral vector comprising a gene for expressing said CAR and a gene for modifying expression of said TNF receptor ligand.

17. A method of enhancing killing of tumor cells in a subject in need thereof, the method comprising administering the genetically modified cell of claim 1 to the subject, whereby killing of tumor cells in the subject is enhanced.

18. The method of claim 17, further comprising producing the genetically modified cell by a method comprising:
(a) harvesting immune cells from a subject;
(b) optionally screening the harvested immune cells for their ability to kill tumor cells of the subject and selecting harvested immune cells having desired function; and
(c) genetically modifying the harvested or screened and selected immune cells for use in anti-tumor therapy for the subject.

19. The method of claim 17, further comprising administering a soluble immune checkpoint inhibitor or a soluble immune modulator to the subject.

20. The method of claim 17, further comprising administering to the subject one or more bispecific antibodies or aptamers having a first binding specificity for a tumor antigen and a second binding specificity for an immune cell.
